(19) United States
(12) Patent Application Publication
(10) Pub. No.: US 2009/0227993 A1
(43) Pub. Date: Sep. 10, 2009

(54) SHAPED FIBER ENDS AND METHODS OF MAKING SAME

(75) Inventor: JING TANG, Arlington, MA (US)

Correspondence Address: MILLS & ONELLO LLP, ELEVEN BEACON STREET, SUITE 605, BOSTON, MA, 02108, US

(73) Assignee: CORNOVA, INC., Burlington, MA (US)

(21) Appl. No.: 12/466,503

(22) Filed: May 15, 2009

Related U.S. Application Data
(63) Continuation-in-part of application No. 12/350,835, filed on Jan. 8, 2009.
(60) Provisional application No. 61/082,721, filed on Jul. 22, 2008.
(60) Provisional application No. 61/025,514, filed on Feb. 1, 2008.
(60) Provisional application No. 61/019,626, filed on Jan. 8, 2008.
(60) Provisional application No. 61/082,721, filed on Jul. 22, 2008.

(51) Int. Cl. A61B 18/18; G02B 6/26; B29D 11/00

(52) U.S. Cl. 606/7; 385/31; 216/24

(57) ABSTRACT

An optical fiber tip comprises a core and a recess formed in said core at a distal end of the optical fiber tip, said recess having a vertex within said core.

### RELATED APPLICATIONS

This application is a continuation-in-part of U.S. patent application Ser. No. 12/350,835, filed on Jan. 8, 2009, the entire contents of which are herein incorporated by reference. This application claims the benefit of U.S. Provisional Application No. 61/082,721 filed on Jul. 22, 2008, the entire contents of which is incorporated herein by reference. This application is related to U.S. Provisional Patent Application No. 61/025,514 filed on Feb. 1, 2008, and U.S. Provisional Application No. 61/019,626 filed Jan. 8, 2008, the entire contents of each of which is incorporated herein by reference. This application is also related to U.S. patent application Ser. No. 11/537,258, filed on Sep. 29, 2006, published as U.S. Patent Application Publication No. 2007/0078500 A1, U.S. patent application Ser. No. 11/834,096, filed on Aug. 6, 2007, published as U.S. Patent Application Publication No. 2007/0270717 A1, and U.S. patent application Ser. No. 12/350,870, filed on Jan. 8, 2009, the contents of each of which is incorporated herein in their entirety by reference.

### BACKGROUND OF THE INVENTION

1. Field of the Invention

Embodiments of the present invention are directed to systems and methods for the analysis and treatment of a lumen. More particularly, embodiments of the present invention relate to a balloon catheter system that is used to perform methods of analysis and angioplasty of endovascular lesions.

2. Description of the Related Art

With the continual expansion of minimally-invasive procedures in medicine, certain procedures that have been highlighted in recent years include catheter applications targeting small tightly curved lumens (e.g., coronary vessels) for diagnosis and treatment or other applications which may benefit from the use of small core diameter fibers (e.g., about 100 microns or less). One type of common procedure is a percutaneous transluminal angioplasty procedure, or “PTA” which, when applied to coronaries, is more specifically called a percutaneous coronary transluminal angioplasty procedure, or “PTCA”. These procedures utilize a flexible catheter with an inflation lumen to expand, under relatively high pressure, a balloon at a distal end of the catheter to expand a stenotic lesion.

The PTA and PTCA procedures are now commonly used in conjunction with expandable tubular structures known as stents and an angioplasty balloon, which is often used to expand and permanently place the stent within the lumen. An angioplasty balloon utilized with a stent is referred to as a stent delivery system. Conventional stents have been shown to be more effective than an angioplasty procedure alone in order to maintain patency in most types of lesions and also to reduce other near-term endovascular events. A risk with a conventional stent, however, is the reduction in efficacy of the stent due to the growth of the tissues surrounding the stent which can again result in the stenosis of the lumen, often referred to as restenosis. In recent years, new stents that are coated with pharmaceutical agents, often in combination with a polymer, have been introduced and shown to significantly reduce the rate of restenosis. These coated stents are generally referred to as drug-eluting stents, though some coated stents have a passive coating instead of an active pharmaceutical agent.

With the advent of these advanced technologies for PTA and PTCA, there has been a substantial amount of clinical and pathology literature published about the pathophysiologic or morphologic factors within an endovascular lesion that contribute to its restenosis or other acute events such as thrombosis. These features include, but are not limited to, collagen content, lipid content, calcium content, inflammatory factors, and the relative positioning of these features within the plaque. Several studies have been provided showing the promise of identifying the above factors through the use of visible and/or near infrared spectroscopy (i.e. across wavelengths ranging between about 250 to 2500 nm), including those studies referenced in U.S. Publication No. US2004/0111016A1 by Casscells, III et al., U.S. Publication No. US2004/0077950A1 by Marshik-Geurts et al., U.S. Pat. No. 5,304,173 by Kittrell et al., and U.S. Pat. No. 6,095,982 by Richards-Kortum, et al., the contents of each of which is herein incorporated by reference. However, there are very few, if any, highly safe and commercially viable applications making use of this spectroscopic data for combining the diagnosis and treatment in a PTA or PTCA procedure. Certain catheter probes, including some described in the aforementioned disclosures, include various therapeutic components but do not combine angioplasty treatments with effective, safe spectroscopic examination and diagnosis with commercially viable flexibility and dimensions for coronary vessel use (e.g., catheters having less than about 1.5 mm in outer diameter and generally having fewer than 8 fibers).

Catheter probes may be small enough and flexible enough for coronary use, but are neverthless very limited in the numbers and dimensions of optical components that can be packaged in the catheter probe's body and distal end. Typical technologies for delivering and/or collecting radiation along a lumen, particularly to and from those target areas peripheral to a catheter body and/or through a peripheral balloon, can require additional features including lenses, reflectors, bent fibers, and the like, which can increase the catheter probe's maximal outer diameter to suboptimal levels for coronary or other small lumen use, add prohibitive costs, and/or are not able to provide an effective and complete analysis of the target coronary vessel region. Some optical fibers developed for smaller probes include shaped ends such as “side-fire” fibers, which have their ends cleaved at an angle and may be subsequently coated so as to direct radiation to or from the fiber tip at a substantial transverse angle. However, these types of fibers still only allow distribution/collection about a limited scope of the periphery of the fiber tip, generally less than about an 83 degree circumferential scope. Shaping the interior profile of optical fiber tips has been proposed such as in, for example, U.S. Pat. No. 5,537,499 by Brekke, the entire contents of which is herein incorporated by reference. Laser and mechanical approaches for fiber-tip formation suggested by such technologies, however, are very impractical and limited for the types of fibers optimal for low profile catheter probes (e.g., with fibers having a core diameters of about 100 microns or less and having maximum outer diameters of about 125 microns or less) because of the necessary precise dimensions of the shaping tool and/or motion required by the shaping tool and/or fiber tip.

### SUMMARY OF THE INVENTION

The systems and methods of the invention provide hospitals and physicians with reliable, simplified, and cost-effective optical components for body lumen inspection devices, including catheter and endoscopic-based devices useful for diagnosing a broad range of tissue conditions. Various embodiments of the invention provide reliable control over multiple light emission paths within a multiple-fiber catheter and/or endoscopic probe while allowing the probe to remain substantially flexible and maneuverable within a body lumen. Reliance on inflexible, expensive, elaborate and/or difficult to assemble components that inhibit prior art devices is thus reduced. By improving control over light emission paths with efficient and low profile components, fewer fibers are required than with typical prior art devices. Thus, improving the flexibility and reducing the size of such a system is especially beneficial for small body vessel applications.

In accordance with an aspect of the invention, there are provided apparatus with fiber optical configurations for performing an optical analysis of a body lumen. In an embodiment, the tips of one or more fibers having maximum core/cladding diameters of 125 microns deliver and/or collect radiation about a circumferential perimeter of the tip of greater than about 90 degrees and, in an embodiment, of greater than about 120 degrees and, in an embodiment, of greater than about 150 degrees and, in an embodiment, of up to 360 degrees. In an embodiment, the tips of the fibers are also manufactured to distribute and/or collect radiation across a longitudinal scope of greater than about 10 degrees in the direction opposite the distal end of the one or more fibers and, in an embodiment, greater than about 30 degrees and, in an embodiment, greater than about 60 degrees. In an embodiment, the tips include a cavity or recess formed out of the terminating end of the tip. In an embodiment, the cavity is conically shaped. In an embodiment, the cavity is elliptically shaped. In an embodiment, the apparatus comprises a lumen-expanding balloon catheter having one or more delivery fibers and/or one or more collection fibers with at least one of a transmission output or a transmission input located within the balloon. In an embodiment, the at least one transmission output or transmission input are held against the inside wall of the balloon such that the transmission output or transmission input will remain proximate to the inside wall of the balloon when the balloon expands.

In an aspect of the invention, the tips of the one or more fibers are modified with a process that forms a cavity or recess or other desired shape in the terminating end of the tip. In an embodiment, the process includes the steps of providing a fiber end with a predetermined core/cladding profile having at least one first material with a first resistance level to an etchant and at least one second material with a second resistance level to the etchant that is greater than the first resistance level. In an embodiment, the concentration of the first material gradually decreases and the concentration of the second material gradually increases as the material's distance from the center of the fiber increases. In an embodiment, the second material comprises silica and the first material comprises a dopant. In an embodiment, the dopant comprises Germanium (Ge). In an embodiment, the dopant comprises at least one of Fluorine (F), Beryllium (Be), and Phosphorous (P). In an embodiment, the etchant comprises Hydrofluoric acid (HF).

In an aspect of the invention, an optical fiber tip comprises a core and a recess formed in said core at a distal end of the optical fiber tip, said recess having a vertex within said core.

In an embodiment, said core has a diameter of about 200 microns or less. In an embodiment, said core has a diameter of about 100 microns or less. In an embodiment, said core has a diameter of about 50 microns or less.

In an embodiment, said core is a graded-index core. In an embodiment, said graded-index core has a material composition with a dopant concentration profile in relation to a shape of said recess. In an embodiment, the dopant concentration profile includes a dopant concentration that has a maximum level at a center of said core. In an embodiment, said maximum level of the dopant concentration at the center of said core is about 15% of the material composition.

In an embodiment, said recess has a shape of a conic section. In an embodiment, said recess has the shape of a cone.

In an embodiment, a cross-section of said recess has a shape of an ellipse. In an embodiment, said recess has a primary vertex located proximal to a center of the core.

In an embodiment, said primary vertex has a maximum depth that is less than a maximum diameter of said core. In an embodiment, said maximum depth is less than 75% of the maximum diameter of said optical fiber tip. In an embodiment, said primary vertex has a maximum depth of less than about 70 microns. In an embodiment, said primary vertex has a maximum depth of less than about 50 microns.

In an embodiment, said recess is covered with at least one of a reflective material, a light diffusing material, and a light blocking material. In an embodiment, said at least one of a reflective material, light diffusing material, and light blocking material comprises at least one of a glass and a polymer. In an embodiment, said at least one of a reflective material, light diffusing material and light blocking material comprises at least one of a thermoplastic and thermosetting plastic. In an embodiment, said at least one of a reflective material, light diffusing, and light blocking material comprises polytetrafluoroethylene.

In an embodiment, the core has a terminating end and wherein an air gap is located between said vertex located within said core and said at least one of the reflective material, light diffusing material, and light blocking material. In an embodiment, said air gap has a span along the longitudinal axis of the fiber tip that is about the same as a width of said core. In an embodiment, said air gap has a span along the longitudinal axis of the fiber tip of about 50 microns or less.

In an embodiment, said tip is manufactured to emit or collect radiation circumferentially around approximately 90 degrees or more of the end of the fiber optics. In an embodiment, said tip is manufactured to emit or collect radiation around approximately 120 degrees or more of the circumference of said tip. In an embodiment, said tip is manufactured to emit or collect radiation around approximately 150 degrees or more of the circumference of said tip. In an embodiment, said tip is manufactured to emit or collect radiation around the entire circumference of said tip.

In another aspect of the invention, a catheter for placement within a body lumen comprises a flexible conduit that elongatedly extends along a longitudinal axis, the flexible conduit having a proximal end and a distal end; and at least one waveguide with a optical fiber tip having a terminating end positioned along the flexible conduit, the optical fiber tip comprising a recess in a terminating end of the optical fiber tip.

In an embodiment, the catheter further comprises a flexible, expandable balloon around said terminating end. In an embodiment, said flexible, expandable balloon is an angioplasty balloon. In an embodiment, said optical fiber tip is radially coupled to said angioplasty balloon.

In another aspect of the invention, a method of manufacturing an optical fiber tip comprises providing an optical fiber core comprising a terminating end; and forming a recess in said terminating end.

In an embodiment, the step of forming a recess comprises applying an etching process to the optical fiber core.

In an embodiment, the method further comprises forming a cladding about said optical fiber core, wherein said optical fiber core and cladding comprises a material composition, the material composition including a first material having a first level of resistance to said etching process and a second material having a second increased level of resistance to said etching process.

In an embodiment, said first material comprises silica.

In an embodiment, said second material comprises germanium. In an embodiment, said second material comprises at least one of germanium, fluorine, beryllium, phosphorous, and hydrofluoric acid.

In an embodiment, across at least a portion of the diameter of said optical fiber and in relation to the distance from the center of said optical fiber core, the concentration of said first material decreases and the concentration of said second material increases in relation to a predetermined shape of said recess.

In an embodiment, the first material is germanium, and the maximum concentration of germanium is about 15% of the material composition at the center of said optical fiber core.

In an embodiment, said optical fiber core comprises a graded index core fiber.

In an embodiment, said optical fiber tip has a core diameter of about 200 microns or less. In an embodiment, said core diameter is about 100 microns or less. In an embodiment, said core diameter is about 50 microns or less.

In an embodiment, said recess is formed in the shape of a conic section. In an embodiment, said recess is formed in the shape of a cone.

In an embodiment, said recess is formed in the shape of an ellipse.

In an embodiment, said recess is formed with a primary vertex located proximal to a center of the core of said optical fiber.

In an embodiment, said primary vertex is formed with a maximum depth from the end of said optical fiber tip that is less than the maximum diameter of the core of said optical fiber tip. In an embodiment, said maximum depth is less than 75% of the maximum diameter of said optical fiber tip.

In an embodiment, said primary vertex is formed with a maximum depth from the end of said optical fiber tip of less than about 70 microns. In an embodiment, said primary vertex is formed with a maximum depth from the end of said optical fiber tip of less than about 50 microns.

In an embodiment, the method further comprises the step of covering said recess with at least one of a reflective material and light diffusing material.

In an embodiment, said at least one of a reflective material and light diffusing material comprises at least one of a glass and a polymer.

In an embodiment, said at least one of a reflective material and light diffusing material comprises at least one of a thermoplastic and thermosetting plastic.

In an embodiment, said at least one of a reflective material and light diffusing material comprises polytetrafluoroethylene.

In an embodiment, the step of covering said recess comprises immersing said optical fiber tip in a solution of said at least one of a reflective material and light diffusing material.

In an embodiment, covering said recess leaves an air gap between a terminating end of the optical fiber core and said at least one of the reflective material and light diffusing material. In an embodiment, said air gap has a span along the longitudinal axis of the fiber tip that is about the same as a width of said core. In an embodiment, said air gap has a span along the longitudinal axis of the fiber tip of about 50 microns or less.

### BRIEF DESCRIPTION OF THE DRAWINGS

The foregoing and other objects, features, and advantages of the invention will be apparent from the more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is an illustrative view of a fiber tip for analyzing and medically treating a lumen, according to an embodiment of the invention.

FIG. 1B is an illustrative cross-sectional view of the fiber tip of FIG. 1A, taken along section lines I-I′.

FIG. 1C is an illustrative view of another fiber tip for analyzing and medically treating a lumen, according to an embodiment of the invention.

FIG. 1D is an illustrative cross-sectional view of the fiber tip of FIG. 1C, taken along section lines II-II′.

FIG. 2A is an illustrative view of a treatment end of a catheter instrument for analyzing and medically treating a lumen according to an embodiment of the present invention.

FIG. 2B is a cross-sectional view of the catheter of FIG. 2A, taken along section lines I-I′ of FIG. 2A.

FIG. 2C is a cross-sectional view of the catheter of FIG. 2A, taken along section lines II-II′ of FIG. 2A.

FIG. 3A is an illustrative view of a catheter instrument for analyzing and medically treating a lumen, according to an embodiment of the present invention.

FIG. 3B is a block diagram illustrating an instrument deployed for analyzing and medically treating the lumen of a patient, according to an embodiment of the present invention.

FIG. 4A is an illustrative schematic view of a fiber tip being formed in an etchant solution according to an embodiment of the invention.

FIG. 4B is an illustrative cross-sectional view of the fiber tip of FIG. 4A, taken along section lines I-I′, while placed in an etchant solution according to an embodiment of the invention.

FIG. 4C is an illustrative schematic view of the fiber tip of FIG. 4A after extraction from an etchant solution.

FIG. 4D is an illustrative schematic view of a portion of an outer protective layer being removed from the fiber tip of FIGS. 4A-4C.

FIG. 5A is an illustrative chart of a dopant concentration of a graded index fiber core in an embodiment of the invention.

FIG. 5B is an illustrative cross-sectional view of a fiber tip formed from a fiber core with a dopant concentration according to the chart of FIG. 5A in an embodiment of the invention.

FIG. 6A is another illustrative chart of dopant concentration of a graded index fiber core in an embodiment of the invention.

FIG. 6B is an illustrative cross-sectional view of a fiber tip formed from a fiber core with a dopant concentration according to the chart of FIG. 6A in an embodiment of the invention.

FIG. 7A is an illustrative cross-sectional view of a fiber tip having an end coated with a reflective material according to an embodiment of the invention.

FIG. 7B is an illustrative perspective view of the fiber tip of FIG. 7A taken along reference line I-I′.

FIG. 7C is an illustrative view of a fiber tip with an air gap spaced between a reflective coating and the core of the tip.

FIG. 8A is an illustrative cross-sectional view of a fiber tip positioned adjacent a reflective surface according to an embodiment of the invention.

FIG. 8B is an illustrative perspective view of the fiber tip and reflective surface of FIG. 8A taken along reference line II-II′.

FIG. 9 is an illustrative perspective view of a fiber tip adjacent a flat reflective surface according to an embodiment of the invention.

FIG. 10 is an illustrative perspective view of a fiber tip adjacent a concave reflective surface according to an embodiment of the invention.

### DETAILED DESCRIPTION OF EMBODIMENTS OF THE INVENTION

The accompanying drawings are described below, in which example embodiments in accordance with the present invention are shown. Specific structural and functional details disclosed herein are merely representative. This invention may be embodied in many alternate forms and should not be construed as limited to example embodiments set forth herein.

Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims. Like numbers refer to like elements throughout the description of the figures.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “on,” “connected to” or “coupled to” another element, it can be directly on, connected to or coupled to the other element or intervening elements may be present. In contrast, when an element is referred to as being “directly on,” “directly connected to” or “directly coupled to” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments and is not intended to be limiting of the invention. As used herein, the singular forms “a,” “an” and “the” are intended to include the plural forms as well, unless the context clearly indicates otherwise. It will be further understood that the terms “comprise,” “comprises,” “comprising,” “include,” “includes” and/or “including,” when used herein, specify the presence of stated features, integers, steps, operations, elements, and/or components, but do not preclude the presence or addition of one or more other features, integers, steps, operations, elements, components, and/or groups thereof.

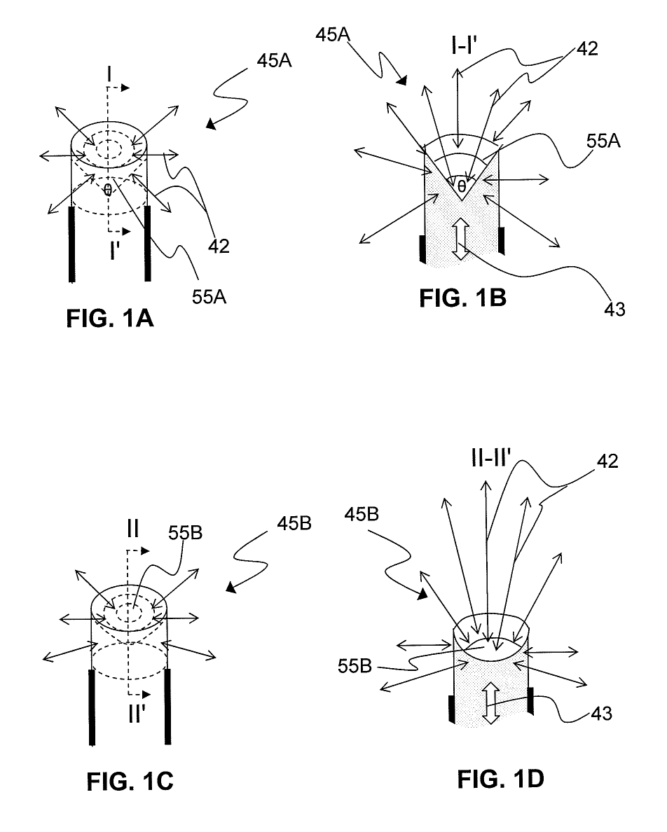

FIG. 1A is an illustrative view of a fiber tip 45A for analyzing and medically treating a lumen, according to an embodiment of the present invention. FIG. 1B is an illustrative cross-sectional view of the fiber tip 45A of FIG. 1A, taken along section lines I-I′. Fiber tip 45A includes a conically-shaped recess 55A formed in a core about which radiation entering and exiting fiber tip 45A may be incident on, such as along exemplary sample trace arrows 42. In an embodiment, fiber tip 45A is adopted as a light delivery/collection end of one or more fibers in an optical probe such as a catheter probe of which embodiments are further described herein. The conically-shaped recess 55A allows radiation to be distributed or collected about a substantially wider directional scope than a conventional fiber end, wherein radiation, for example, optical radiation such as light (e.g., along trace lines 42) is refracted or reflected at various angles after becoming incident upon the recess 55A. In other embodiments, the recess 55A can have other shapes, such that a vertex is located within the core of the tip 45A. In other embodiments, recess 55A can have other shapes that comprise higher order polynomial curves. In other embodiments, the recess has a curved surface, the curved surface having a vertex within the core.

A fiber with a recessed tip in accordance with an embodiment of the invention permits the recess 55A to allow light 43 passing through the fiber in a direction of the fiber to be collected from or distributed or otherwise redirected in directions substantially transverse to the direction of the light 43 passing through the fiber. For example, the angle θ defining the conical shape of recess 55A can be increased so as to allow distribution and/or collection of radiation across a range of directions relative to the longitudinal direction of the fiber, for example, the directions being greater than about 10 degrees and up to about 120 degrees off-axis from the longitudinal axis of the fiber. The conically-shaped recess 55A also allows light to be distributed/collected up to a full 360 degree periphery about the fiber tip circumference. In various embodiments, the fibers with recesses in accordance with those described herein have cores with maximum diameters of about 100 microns or less (and total maximum outer diameters of 125 microns or less). These embodiments thereby significantly increase the effective numerical aperture and control over transmission to/from low diameter fibers without the need for bending the fiber and/or adding separate optical components such as, for example, lenses, reflectors, and the like.

The shape of a recess of a fiber tip in accordance with an embodiment of the invention can be configured in order to provide a particular distribution/collection profile. For example, FIG. 1C is an illustrative view of another fiber tip for analyzing and medically treating a lumen, according to an embodiment of the present invention. FIG. 1D is an illustrative cross-sectional view of the fiber tip of FIG. 1C, taken along section lines II-II′. Accordingly, the shape of the recess of the fiber tip shown in FIGS. 1C and 1D is different than the conical shape of FIGS. 1A and 1B, permitting the fiber tip shown in FIGS. 1C and 1D to correspond to a different distribution/collection profile. However, the recess 55B shown in FIGS. 1C and 1D can have other shapes with a recess having a vertex located within the core of the tip 45B. In other embodiments, recess 55B can have other shapes that comprise higher order polynomial curves. In other embodiments, the recess 55B has a curved surface, the curved surface having a vertex within the core. In an embodiment, a recess 55B is configured in an elliptically-shaped manner which can allow more light to be distributed between the longitudinal/side direction than that of a more angularly sharper recess (e.g., such as that of FIGS. 1A-1B). In an embodiment, a fiber tip recess is adapted in relation to a fiber's core/cladding components to provide a desired optical profile such as, for example, those described in further detail herein below.

Formed tips according to various embodiments of the invention can increase the directional scope (aperture) in which light is delivered and collected and, in particular, those directions transverse to the longitudinal axis of the catheter's treatment end. The formed tips are particularly beneficial for near-field type scanning around the circumferential periphery of the tips and, in an embodiment, are adapted for use in fibers that are maintained in close peripheral contact to the outside edge of an angioplasty-type balloon system such as described further herein. The embodiment is particularly advantageous in that it may avoid the need for many of the additional components (e.g., reflectors, lenses, etc. . . . ) common to typical optical fiber catheter probes while allowing for delivery and collection of radiation across a wide area. In an embodiment, the potential loss of power associated with the removal of a core and cladding from the fiber is mitigated by the close proximity in which various embodiments position the tips 45A, 45B in relation to targeted tissue and/or fluids

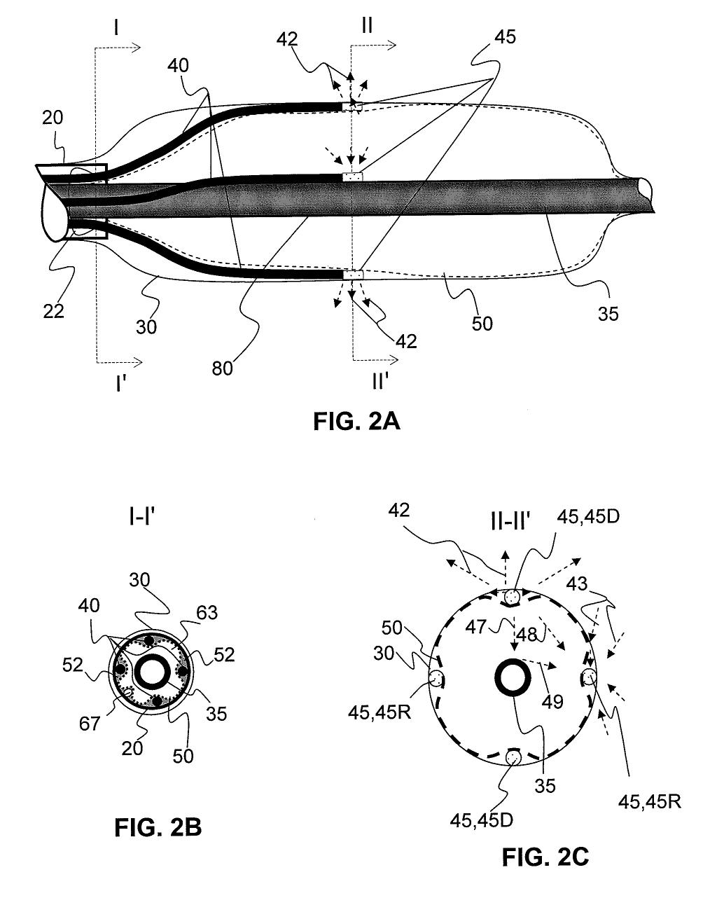

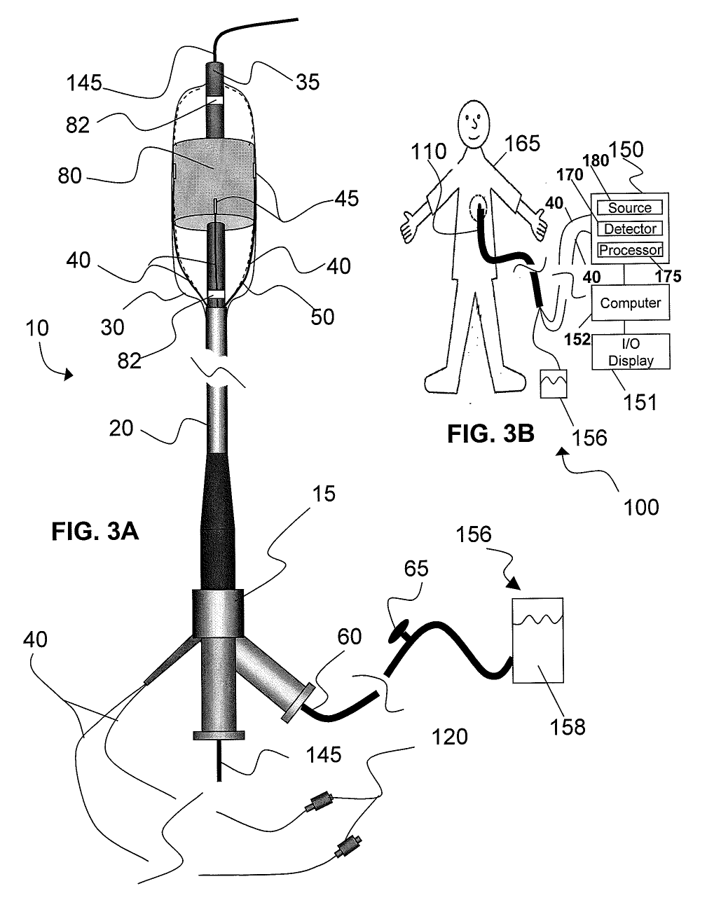

FIG. 2A is an expanded illustrative view of the treatment end of a catheter instrument incorporating fiber tips 45 in accordance with an embodiment of the present invention. FIG. 2B is a cross-sectional view of the catheter of FIG. 2A, taken along section lines I-I′ of FIG. 2A. FIG. 2C is a cross-sectional view of the catheter of FIG. 2A, taken along section lines II-II′ of FIG. 2A. In an embodiment, a flexible outer covering 30 can operate as an inflatable balloon and is attached at its proximal end about a catheter sheath 20. An inner balloon 50, fibers 40, and a guidewire sheath 35 extend through an opening 22 at a distal end of catheter sheath 20 and into inner balloon 50. In an embodiment, a proximal end of inner balloon 50 is attached to the interior of catheter sheath 20 with glue 52 placed between inner balloon 50 and catheter sheath 20. An intervening lumen 63 formed between catheter sheath 20 and guidewire sheath 35 can be used to transfer fluid media to inner balloon 50 from a fluid source (e.g., liquid/gas source 156 of FIGS. 3A-3B). A separate lumen 67 can be used to transfer fluid to and from the area between outer covering 30 and inner balloon 50 (e.g., as in an angioplasty balloon).

In an embodiment, both inner balloon 50 and lumen 67 are supplied simultaneously by the same fluid source. Inner balloon 50 is initially filled with fluid and will continue to expand against outer covering 30 as fluid pressure between inner balloon 50 and guidewire sheath 35 and the fluid pressure between the outer covering 30 and inner balloon 50 equalize, resulting in the distal end acting as an angioplasty balloon while substantially maintaining the delivery and collection ends 45 of fibers 40 against the inside wall of outer covering 30. Fiber tips 45 can be in accordance with, for example, those of FIGS. 1A-1D so as to allow distribution and/or collection of radiation (e.g., along exemplary trace lines 42) about the periphery of outer covering 30 and an adjacent lumen wall. In an embodiment, fiber tips 45 include two delivery ends 45D for delivering radiation and two collection ends 45R for receiving radiation.

In an embodiment, radiation can also be directed/collected between fiber tips 45 by way of the balloon interior (e.g., along exemplary trace lines 47, 48, and 49) so as to obtain and monitor information about the distance between fiber tips 45 (and balloon 30) and sheath 35 and thus provide information about the level and uniformity of expansion of balloon 30. In an embodiment, preliminary readings are taken of signals received through light reflected from sheath 35 and the corresponding measured sizes of balloon 30. This information can then later be used during deployment to provide estimates of the level of expansion of balloon 30. In an embodiment, a source/type of radiation of a wavelength range distinct from that used for examining the lumen wall is used to monitor the level of expansion of balloon 30. In an embodiment, the sheath 35 can include material coating so as to reflect, enhance, and/or modify signals directed to the sheath from fiber tips 45, after which a distinct signal is received corresponding to the level of expansion of balloon 30. In an embodiment, inner balloon 50 may include a reflective coating (e.g., as shown and described in reference to FIG. 3A) for aiding in the distribution and collection of radiation between fiber ends 45 and the lumen wall. In an embodiment, the reflective coating can be manufactured to allow selected radiation to pass through (e.g., as in a bandpass filter or through a small gap in the reflective coating) toward sheath 35.

FIG. 3A is an illustrative view of a catheter instrument 10 for analyzing and medically treating a lumen, according to an embodiment of the present invention. FIG. 3B is a block diagram illustrating an instrument 100 deployed for analyzing and medically treating the lumen of a patient, according to an embodiment of the present invention. The catheter assembly 10 includes a catheter sheath 20 and at least two fibers 40, including one or more delivery fiber(s) connected to at least one source 180 and one or more collection fiber(s) connected to at least one detector 170. Catheter sheath 20 includes a guidewire sheath 35 and guidewire 145. The distal end of catheter assembly 10 includes an inner balloon 50 and a flexible outer covering 30. In an embodiment, inner balloon 50 and outer covering 30 function as a lumen expanding balloon (e.g., an angioplasty balloon).

Delivery and collection ends 45 of fibers 40 are positioned between the inner balloon 50 and outer covering 30. Inner balloon 50 can include a reflective surface 80 facing outwardly so as to improve light delivery and collection to and from delivery/collection ends 45. The reflective surface 80 can be applied, for example, as a thin coating of reflective material such as gold paint or laminate or other similar material known to those of skill in the art. Outer covering 30 is comprised of a material translucent to radiation delivered and collected by fibers 40 such as, for example, translucent nylon or other polymers. The delivery and collection ends 45 are preferably configured to deliver and collect light about a wide angle such as, for example, between about at least a 120 to 180 degree cone around the circumference of each fiber, from a direction outward toward targeted tissues/fluids such as exemplified in FIGS. 1A-1D and 2C. Various methods for forming such delivery and collection ends are described in more detail herein below. Various such embodiments in accordance with the invention allow for diffusely reflected light to be readily delivered and collected between fibers 40 and tissue surrounding the distal end of catheter 10.

The proximate end of balloon catheter assembly 10 includes a junction 15 that connects various conduits between catheter sheath 20 to external system components. Fibers 40 can be fitted with connectors 120 (e.g. FC/PC type) compatible for use with light sources, detectors, and/or analyzing devices such as spectrometers. Two radiopaque marker bands 82 are fixed about guidewire sheath 35 in order to help an operator obtain information about the general location of catheter 10 in the body of a patient (e.g. with the aid of a fluoroscope).

The proximal ends of fibers 40 are connected to a light source 180 and/or a detector 170 (which are shown integrated with an analyzer/processor 150). Analyzer/processor 150 can be, for example, a spectrometer which includes a processor 175 for processing/analyzing data received through fibers 40. A computer 152 connected to analyzer/processor 150 can be used to operate the instrument 100 and to further process spectroscopic data (including, for example, through chemometric analysis) in order to diagnose and/or treat the condition of a subject 165. Input/output components (I/O) and viewing components 151 are provided in order to communicate information between, for example, storage and/or network devices and the like and to allow operators to view information related to the operation of the instrument 100.

Various embodiments provide a spectrometer (e.g., as analyzer/processor 150) configured to perform spectroscopic analysis within a wavelength range between about 250 and 2500 nanometers and include embodiments having ranges particularly in the near-infrared spectrum between about 750 and 2500 nanometers. Further embodiments are configured for performing spectroscopy within one or more subranges that include, for example, about 250-930 nm, about 1100-1385 nm, about 1600-1850 nm, and about 2100-2500 nm. Various embodiments are further described in, for example, previously cited and co-pending U.S. application Ser. No. 11/537,258 (entitled “SYSTEMS AND METHODS FOR ANALYSIS AND TREATMENT OF A BODY LUMEN”), and U.S. application Ser. No. 11/834,096 (entitled “MULTI-FACETED OPTICAL REFLECTOR”), the entire contents of each of which is herein incorporated by reference.

Junction 15 includes a flushing port 60 for supplying or removing fluid media (e.g., liquid/gas) 158 that can be used to expand or contract inner balloon 50 and, in an embodiment, an outer balloon formed by flexible outer covering 30. Fluid media 158 is held in a tank 156 from which it is pumped in or removed from the balloon(s) by actuation of a knob 65. Fluid media 158 can alternatively be pumped with the use of automated components (e.g. switches/compressors/vacuums). Solutions for expansion of the balloon are preferably non-toxic to humans (e.g. saline solution) and are substantially translucent to the selected light radiation.

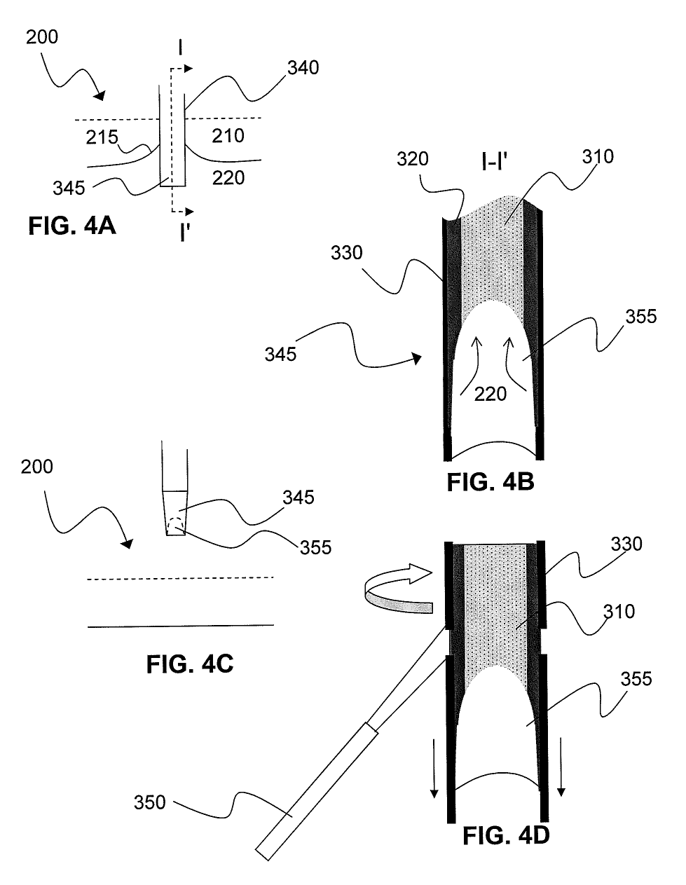

FIG. 4A is an illustrative schematic view of a fiber tip being formed in an etchant solution according to an embodiment of the invention. FIG. 4B is an illustrative cross-sectional view of the fiber tip of FIG. 4A, taken along section lines I-I′, while placed in an etchant solution according to an embodiment of the invention. FIG. 4C is an illustrative schematic view of the fiber tip of FIG. 4A after extraction from an etchant solution. FIG. 4D is an illustrative schematic view of a portion of the outer protective layer being removed from the fiber tip of FIGS. 4A-4C.

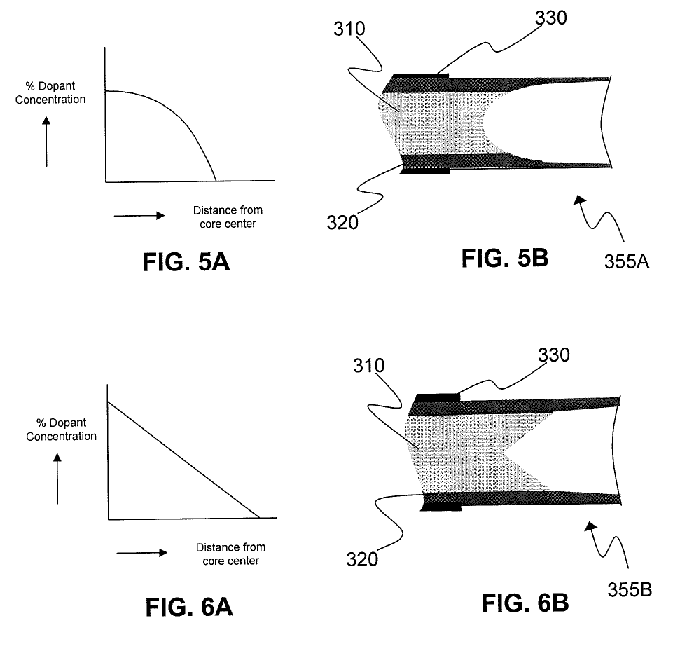

In an embodiment, the process for forming a fiber tip 345 occurs (as shown in FIG. 4A) by placing the end of a fiber 340 in a bath 200 including an etchant 220. Fiber tip 345 includes a core 310, a cladding layer 320, and a protective outer layer 330. In an embodiment, the etchant 220 comprises Hydrofluoric Acid (HF). An organic solvent 210 (e.g., silicone) can be included in the bath so as to control formation of a meniscus 215 and to prevent inadvertent exposure of portions of fiber 340 to the etchant. In an embodiment, a second material of the fiber tip such as pure silicon has a level of resistance to the etchant 220 and a first material such as a dopant (e.g., germanium) has a different level of resistance to the etchant. In an embodiment, portions of the core have different resistance levels to the etchant, the resistance levels of portions of the core dependent on the concentrations of the first and second materials. For example, a portion of the fiber tip 345 proximal to the center of the fiber tip can comprise approximately 15% of the first material, where the fiber tip 345 has the least amount of resistance to the etchant, and a portion of the fiber tip 345 proximal to an outer surface of the fiber tip can comprise approximately 0% of the first material, where the fiber tip has the greatest resistance to the etchant. Depending on the fiber type and the desired profile/shape of tip 345 (e.g., such as those shown and described in reference to FIGS. 5-6), the materials of a first and second resistance can be mixed at different concentrations within different locations of the core. In an embodiment, the core 310 is formed utilizing a process such as activated chemical vapor deposition such that fine layers of core material are applied about the circumference of the core 310 with the desired concentrations of first and second materials (e.g., dopant and silica, respectively,) for providing the desired resistance levels relative to specific distances from the center of the core. In an embodiment, a layer of the core material applied during the process is about 0.004 inches thick. Fiber 340 is shown held in bath 200 of etchant solution for a predetermined amount of time. In an embodiment, fiber 340 has a graded index core with a diameter of between about 50 and 100 microns and is held in the etchant 220 for a period between about 4 minutes to 15 minutes or more. Fiber 340 can also be moved and repositioned in the etchant to affect the shape of tip 345. As illustrated in FIG. 4B, etchant solution 220 gradually removes material from the cladding/core interior of fiber tip 345, forming a shaped recess 355 within the cladding/core interior. In various embodiments, general techniques for applying etchant solutions to fiber tips for forming pointed or sharpened ends are adapted for forming recessed tips as described herein. Some techniques for etching pointed or sharpened tip ends are described in P. K. Wong et al., “Optical Fiber Tip Fabricated By Surface Tension Controlled Etching,” CM Ho—Proc. of Hilton Head (2002), Lazarev, et al., “Formation of fine near-field scanning optical microscopy tips. Part I. By static and dynamic chemical etching,” Rev. Sci. Instrum. 74, 3684 (2003), U.S. Pat. No. 6,905,623 by Wei at al., the entire contents of each of which is herein incorporated by reference.

After application of the etchant solution 220 to tip 345 to form the desired shape of the recess 355, fiber tip 345 is removed from the solution (as shown in FIG. 4C) and subsequently cleaned of etchant and solvent. In an embodiment, the tip can be additionally polished so as to remove imperfections along the outer periphery of the fiber tip.

In an embodiment, the outer protective layer 330 is removed from a portion of tip 345 so as to allow radiation to travel between the core of fiber 340 and locations transverse the longitudinal axis of fiber tip 345. In an embodiment, the removal process uses a laser 350 (as shown in FIG. 4D) to cut a thin slice through layer 330, after which the portion 330′ of layer 330 distal to the slice can be removed from tip 345, as shown by arrows. In various embodiments, laser, chemical, and/or mechanical processes known to those of ordinary skill in the art can be used to remove the portion 330′ of outer layer 330 without undue damage to the interior core/cladding of fiber tip 345.

In an embodiment, the formed tips are applied to fibers having graded index cores with maximum core diameters of about 100 microns or less and, in an embodiment, are of about 50 microns or less. In an embodiment, the maximum outer diameters of the fibers are of about 125 microns or less and in an embodiment, are of about 70 microns or less with appropriately sized layers of cladding and protective outer material (e.g., polyimide). Fibers with preferable core sizes between about 50 to 100 microns in various embodiments of the invention can be facilitated with generally thinner than typical overcladding/protective layers because the fibers will generally remain highly protected within the catheter components such as those described herein. Fibers with cores having diameters as small as about 9 microns for use with various embodiments of the invention can be obtained with various requested properties (e.g., low profile overcladding/jackets, doping profiles) from, for example, Yangtze Optical Fiber and Cable Co., Ltd. of Wuhan, China (See http://www.yofcfiber.com) and OFS Specialty Photonics (See http://www.specialtyphotonics.com) having offices in Avon, Conn. and Somerset, N.J., and/or manufactured in accordance with various known methods such as, for example, those described in U.S. Pat. No. 7,013,678, U.S. Pat. No. 6,422,043, and U.S. Pat. No. 5,774,607, the contents of each of which is herein incorporated by reference.

A dopant that can be used in a graded-index embodiment of the invention comprises Germanium (Ge) while the remaining component of a fiber consists essentially of silica. In an embodiment, the dopant comprises at least one of Fluorine (F), Beryllium (Be), and Phosphorous (P). In an embodiment, the change in the index of refraction across the diameter of the fiber core ranges between about 1.458 and 1.49 wherein the maximum index of refraction occurs in the center of the fiber core and varies approximately in proportion to the dopant concentration. In an embodiment, the maximum dopant concentration is about 15% of the material composition of the fiber at the center of a fiber and is gradually reduced to a concentration of 0% of the material composition of the fiber, for example, as presented in FIGS. 5A and 6A.

FIG. 5A is an illustrative chart of the dopant concentration of a graded index fiber core in an embodiment of the invention. In an embodiment, the dopant concentration is configured to provide an etched core including the shape of a conic section (i.e., that of the intersection between a plane and a cone). For example, FIG. 5B is an illustrative cross-sectional view of a fiber tip 355A formed from a graded index fiber core with a dopant concentration having an elliptical profile such as according to the chart of FIG. 5A. In an embodiment of the invention, a wet etching process such as described above is applied to form the fiber tip 355A and produce a recess within the core having cross-sections in the shape of an ellipse.

FIG. 6A is another illustrative chart of dopant concentration of a graded index fiber core in another embodiment of the invention. FIG. 6B is an illustrative cross-sectional view of a fiber tip 355B formed from a fiber core with a dopant concentration having a linear profile such as according to the chart of FIG. 6A. In an embodiment of the invention, a wet etching process such as described above is applied to a fiber tip so as to provide a cone-shaped shaped recess 355B.

The core's graded indexing can be adjusted to provide a particular desired optical configuration. In various embodiments of the invention, the fiber tip can be cleaved at various angles prior to etching so as to also help configure the tip to a desired optical configuration (e.g., and help concentrate delivered/collected radiation along various axis).

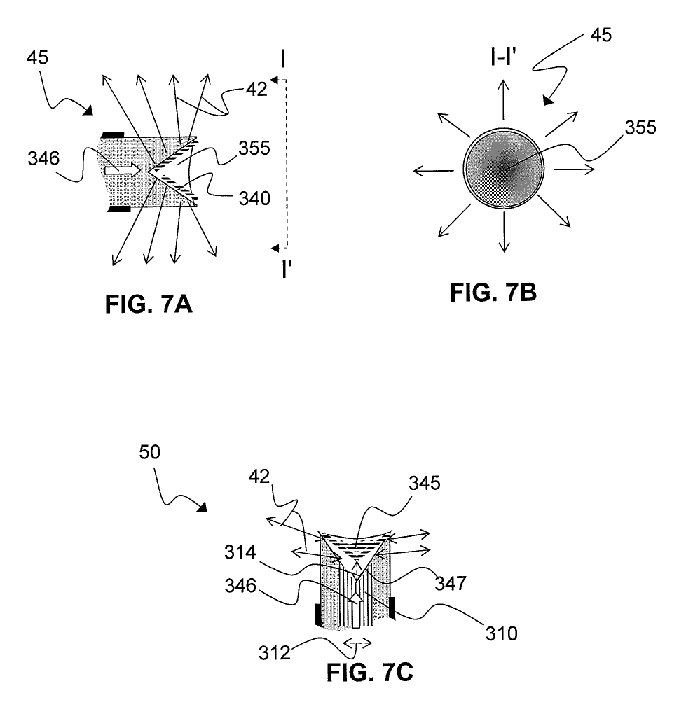

FIG. 7A is an illustrative cross-sectional view of a fiber tip having an end coated with a reflective and/or light diffusing material according to an embodiment of the invention. FIG. 7B is an illustrative perspective view of the fiber tip of FIG. 7A taken along reference line I-I′. A coating 340 is added to the recess 355, which promotes distribution/collection of radiation along various axes transverse to the longitudinal axis of fiber tip 45. The coating 340 can be added by applying a reflective (e.g., gold, silver) spray coating to recessed surface of the tip 45 (after masking off the other surfaces of tip 45) or filling in the recess with a reflective material such as a highly reflective polymer or metallic material including, for example, those that can be shaped/molded and/or later hardened with curing. In an embodiment, the reflective material is applied prior to removal of an outer protective jacket (e.g., jacket 330, 330′ of FIGS. 4B and 4D). In this manner, the jacket may serve to protect aspects of the tip 345 from contamination by the coating 340.

FIG. 7C is an illustrative view of a fiber tip 50 with an air gap 347 spaced between a reflective coating 345 and the core 310 of the tip 50. In an embodiment, such an air gap 347 provides a greater change between indices of refraction across the outer boundary of the core 310 where light enters or exits, thus increasing the level light is directed off-axis from the longitudinal path 346 of the fiber core 310. In an embodiment, the width 312 of the gap is approximately the width of the fiber core 310. In an embodiment, the height 314 of the gap is approximately the same as the width of the fiber core 310. In an embodiment, the width 312 and height 314 of the gap 3 are about 50 microns or less.

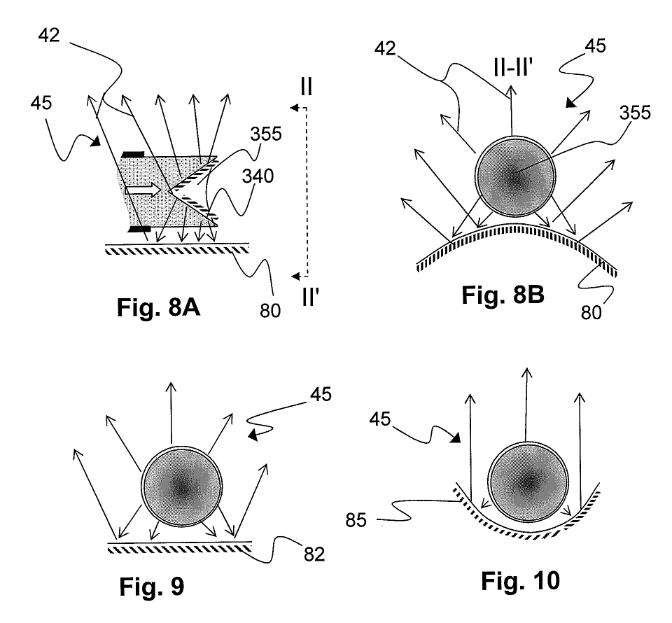

FIG. 8A is an illustrative cross-sectional view of a fiber tip 45 positioned adjacent a reflective surface 80 according to an embodiment of the invention. FIG. 8B is an illustrative perspective view of the fiber tip 45 and reflective surface 80 of FIG. 8A taken along reference line II-II′. In an embodiment, a reflective surface 80 is placed adjacent a fiber tip 45 so that tip 45 is positioned between reflective surface 80 and targeted body tissue/fluids such as those described herein with regard to FIG. 3A. Placement of surface 80 in this manner can help direct more radiation between tip 45 and targeted body tissue/fluids. In an embodiment, a small translucent area can be made in surface 80 so as to allow some radiation to pass between tip 45 and inner components of a catheter such as exemplary transmission paths 47, 48, and 49 shown in FIG. 2C. In an embodiment, the reflective surface is shaped in a convex manner with respect to outside body tissue/fluids (as shown in FIG. 8B) so as to allow a wider circumferential scope of radiation to be delivered/collected.

FIG. 9 is an illustrative perspective view of a fiber tip 45 adjacent a flat reflective surface 82 according to an embodiment of the invention. A flatter surface can concentrate the scope of delivered/collected radiation in a bearing more direct to body tissue/fluids than a convex surface would. In an embodiment, one or more customized distinct reflective surfaces can be arranged adjacent to individual fiber tips such as flat rectangular pieces attached to an inner balloon (e.g., see co-pending U.S. Application No. 61/019,626, filed on Jan. 8, 2008, the entire contents of which has been incorporated by reference above). FIG. 10 is an illustrative perspective view of a fiber tip 45 adjacent a concave reflective surface 85 according to another embodiment of the invention. A more concave surface with respect to bodily tissue/fluids can help concentrate and/or evenly distribute radiation directed between a fiber tip 45 and the targeted tissue/fluids.

It will be understood by those with knowledge in related fields that uses of alternate or varied forms or materials and modifications to the methods disclosed are apparent. This disclosure is intended to cover these and other variations, uses, or other departures from the specific embodiments as come within the art to which the invention pertains.

What is claimed is:

1. An optical fiber tip comprising:
a core; and
a recess formed in said core at a distal end of the optical fiber tip, said recess having a vertex within said core.

2. The optical fiber tip of claim 1 wherein said core has a diameter of about 100 microns or less.

3. The optical fiber tip of claim 2 wherein said core has a diameter of about 50 microns or less.

4. The optical fiber tip of claim 1 wherein said core is a graded-index core.

5. The optical fiber tip of claim 4 wherein said graded-index core has a material composition having a dopant concentration profile in relation to a shape of said recess.

6. The optical fiber tip of claim 5 wherein the dopant concentration profile includes a dopant concentration that has a maximum level at a center of said core.

7. The optical fiber tip of claim 6 wherein said maximum level of the dopant concentration at the center of said core is about 15% of the material composition.

8. The optical fiber tip of claim 1 wherein said recess has a shape of a conic section.

9. The optical fiber tip of claim 8 wherein said recess has a shape of a cone.

10. The optical fiber tip of claim 8 wherein a cross-section of said recess has a shape of an ellipse.

11. The optical fiber tip of claim 1 wherein said recess has a primary vertex located proximal to a center of the core.

12. The optical fiber tip of claim 11 wherein said primary vertex has a maximum depth that is less than a maximum diameter of said core.

13. The optical fiber tip of claim 12 wherein said maximum depth is less than 75% of the maximum diameter of said optical fiber tip.

14. The optical fiber tip of claim 11 wherein said primary vertex has a maximum depth of less than about 70 microns.

15. The optical fiber tip of claim 11 wherein said primary vertex has a maximum depth of less than about 50 microns.

16. The optical fiber tip of claim 1 wherein said recess is covered with at least one of a reflective material, a light diffusing material, and a light blocking material.

17. The optical fiber tip of claim 16 wherein said at least one of the reflective material, light diffusing material, and light blocking material comprises at least one of a glass and a polymer.

18. The optical fiber tip of claim 16 wherein said at least one of the reflective material, light diffusing material and light blocking material comprises at least one of a thermoplastic and thermosetting plastic.

19. The optical fiber tip of claim 18 wherein said at least one of the reflective material, light diffusing, and light blocking material comprises polytetrafluoroethylene.

20. The optical fiber tip of claim 16 wherein the core has a terminating end and wherein an air gap is located between said vertex located within said core and said at least one of the reflective material, light diffusing material, and light blocking material.

21. The optical fiber tip of claim 20 wherein said air gap has a span along the longitudinal axis of the fiber tip that is about the same as a width of said core.

22. The optical fiber tip of claim 20 wherein said air gap has a span along the longitudinal axis of the fiber tip of about 50 microns or less.

23. The optical fiber tip of claim 1 wherein said tip is manufactured to emit or collect radiation circumferentially around approximately 90 degrees or more of the end of the fiber optics.

24. The optical fiber tip of claim 1 wherein said tip is manufactured to emit or collect radiation around approximately 120 degrees or more of the circumference of said tip.

25. The optical fiber tip of claim 24 wherein said tip is manufactured to emit or collect radiation around approximately 150 degrees or more of the circumference of said tip.

26. The optical fiber tip of claim 25 wherein said tip is manufactured to emit or collect radiation around the entire circumference of said tip.

27. A catheter for placement within a body lumen, the catheter comprising:
a flexible conduit that elongatedly extends along a longitudinal axis, the flexible conduit having a proximal end and a distal end; and
at least one waveguide with a optical fiber tip having a terminating end positioned along the flexible conduit, the optical fiber tip comprising a recess in a terminating end of the optical fiber tip.

28. The catheter of claim 27 further comprising a flexible, expandable balloon around said terminating end.

29. The catheter of claim 28 wherein said flexible, expandable balloon is an angioplasty balloon.

30. The catheter of claim 29 wherein said optical fiber tip is radially coupled to said angioplasty balloon.

31. A method of manufacturing an optical fiber tip, the method comprising:
providing an optical fiber core comprising a terminating end; and
forming a recess in said terminating end.

32. The method of claim 31 wherein the step of forming a recess comprises applying an etching process to the optical fiber core.

33. The method of claim 32 further comprising forming a cladding about said optical fiber core, wherein said optical fiber core and cladding comprises a material composition, the material composition including a first material having a first level of resistance to said etching process and a second material having a second increased level of resistance to said etching process.

34. The method of claim 33 wherein said second material comprises silica.

35. The method of claim 33 wherein said first material comprises at least one of germanium, fluorine, beryllium, phosphorous, and hydrofluoric acid.

36. The method of claim 33 wherein across at least a portion of the diameter of said optical fiber and in relation to the distance from the center of said optical fiber core, the concentration of said first material decreases and the concentration of said second material increases in relation to a predetermined shape of said recess.

37. The method of claim 36 wherein said first material is germanium, and wherein a maximum concentration of germanium is about 15% of the material composition at the center of said optical fiber core.

38. The method of claim 31 wherein said optical fiber core comprises a graded index core fiber.

39. The method of claim 30 wherein said core diameter is about 100 microns or less.

40. The method of claim 38 wherein said core diameter is about 50 microns or less.

41. The method of claim 31 wherein said recess is formed in a shape of a conic section.

42. The method of claim 41 wherein said recess is formed in a shape of a cone.

43. The method of claim 41 wherein said recess is formed in a shape of an ellipse.

44. The method of claim 31 wherein said recess is formed with a primary vertex located proximal to a center of the core of said optical fiber.

45. The method of claim 44 wherein said primary vertex is formed with a maximum depth from the end of said optical fiber tip that is less than the maximum diameter of the core of said optical fiber tip.

46. The method of claim 45 wherein said maximum depth is less than 75% of the maximum diameter of said optical fiber tip.

47. The method of claim 31 wherein said primary vertex is formed with a maximum depth from the end of said optical fiber tip of less than about 70 microns.

48. The method of claim 31 wherein said primary vertex is formed with a maximum depth from the end of said optical fiber tip of less than about 50 microns.

49. The method of claim 31 further comprising the step of covering said recess with at least one of a reflective material and light diffusing material.

50. The method of claim 49 wherein said at least one of a reflective material and light diffusing material comprises at least one of a glass and a polymer.

51. The method of claim 49 wherein said at least one of a reflective material and light diffusing material comprises at least one of a thermoplastic and thermosetting plastic.

52. The method of claim 51 wherein said at least one of a reflective material and light diffusing material comprises polytetrafluoroethylene.

53. The method of claim 49 wherein the step of covering said recess comprises immersing said optical fiber tip in a solution of said at least one of a reflective material and light diffusing material.

54. The method of claim 49 wherein covering said recess leaves an air gap between a terminating end of the optical fiber core and said at least one of the reflective material and light diffusing material.

55. The optical fiber tip of claim 54 wherein said air gap has a span along the longitudinal axis of the fiber tip that is about the same as a width of said core.

56. The optical fiber tip of claim 54 wherein said air gap has a span along the longitudinal axis of the fiber tip of about 50 microns or less.
